(19) United States
(12) Patent Application Publication
(10) Pub. No.: US 2023/0293247 A1
(43) Pub. Date: Sep. 21, 2023

(54) MODULAR APPARATUS FOR ROBOT-ASSISTED ELECTROSURGERY

(71) Applicant: Creo Medical Limited, Chepstow, Monmouthshire (GB)

(72) Inventors: Christopher Paul Hancock, Chepstow (GB); Simon Meadowcroff, Chepstow (GB); John Bishop, Chepstow (GB); George Christian Ullrich, Bethesda (GB)

(73) Assignee: Creo Medical Limited, Chepstow, Monmouthshire (GB)

(21) Appl. No.: 18/020,066

(22) PCT Filed: Jul. 14, 2021
(86) PCT No.: PCT/EP2021/069652

§ 371 Date: Feb. 6, 2023

(30) Foreign Application Priority Data
Aug. 7, 2020 (GB) 2012303.0

(51) Int. Cl. A61B 34/30; A61B 18/12; A61B 18/18; A61B 17/00

(52) U.S. Cl. CPC A61B 34/30; A61B 18/1206; A61B 18/1815; A61B 17/00; A61B 2034/305; A61B 2017/00477; A61B 2018/00011

(57) ABSTRACT

A robot-assisted surgical system in which apparatus for providing electrosurgical functionality is directly mountable on or integrated within a robotic arm. The apparatus may be a detachable module or capsule, which may be movable between different robotic arms in the same environment. The apparatus may comprise a plurality of modules, each providing a different treatment modality. Depending on the procedure to be performed, a different module or combination of modules may be selected and mounted on one or more robotic arms.

### FIELD OF THE INVENTION

The invention relates to apparatus for robot-assisted electrosurgery. In particular, the invention relates to various modules that can be incorporated into a robotic surgery system to enable that system to operate with electrosurgical instruments. The modules may be detachably mountable, e.g. to permit them to be exchanged between different robotic systems within the same operating theatre environment. The modules may be capable of retrofitting to existing robotic surgery systems.

The modules may generate various types of electromagnetic energy for use in electrosurgical instrument. For example, radiofrequency and/or microwave energy may be generated to treat or measure biological tissue. For example, radiofrequency and/or microwave energy may be used to perform any of ablation, haemostasis (i.e. sealing broken blood vessels by promoting blood coagulation), cutting, sterilization, etc.

### BACKGROUND TO THE INVENTION

Electromagnetic (EM) energy, and in particular microwave and radiofrequency (RF) energy, has been found to be useful in electrosurgical operations, for its ability to cut, coagulate, and ablate body tissue. Typically, apparatus for delivering EM energy to body tissue includes a generator comprising a source of EM energy, and an electrosurgical instrument connected to the generator, for delivering the energy to tissue.

Tissue ablation using microwave EM energy is based on the fact that biological tissue is largely composed of water. Human soft organ tissue is typically between 70% and 80% water content. Water molecules have a permanent electric dipole moment, meaning that a charge imbalance exists across the molecule. This charge imbalance causes the molecules to move in response to the forces generated by application of a time varying electric field as the molecules rotate to align their electric dipole moment with the polarity of the applied field. At microwave frequencies, rapid molecular oscillations result in frictional heating and consequential dissipation of the field energy in the form of heat. This is known as dielectric heating.

This principle is harnessed in microwave ablation therapies, where water molecules in target tissue are rapidly heated by application of a localised electromagnetic field at microwave frequencies, resulting in tissue coagulation and cell death. It is known to use microwave emitting probes to treat various conditions in the lungs and other organs. For example, in the lungs, microwave radiation can be used to treat asthma and ablate tumours or lesions.

Surgical resection is a means of removing sections of organs from within the human or animal body. Such organs may be highly vascular. When tissue is cut (divided or transected) small blood vessels called arterioles are damaged or ruptured. Initial bleeding is followed by a coagulation cascade where the blood is turned into a clot in an attempt to plug the bleeding point. During an operation, it is desirable for a patient to lose as little blood as possible, so various devices have been developed in an attempt to provide blood free cutting.

Instead of a sharp blade, it is known to use radiofrequency (RF) energy to cut biological tissue. The method of cutting using RF energy operates using the principle that as an electric current passes through a tissue matrix (aided by the ionic contents of the cells and the intercellular electrolytes), the impedance to the flow of electrons across the tissue generates heat. When an RF voltage is applied to the tissue matrix, enough heat is generated within the cells to vaporise the water content of the tissue. As a result of this increasing desiccation, particularly adjacent to the RF emitting region of the instrument (referred to herein as an RF blade) which has the highest current density of the entire current path through tissue, the tissue adjacent to the cut pole of the RF blade loses direct contact with the blade. The applied voltage then appears almost entirely across this void which ionises as a result, forming a plasma, which has a very high volume resistivity compared to tissue. This differentiation is important as it focusses the applied energy to the plasma that completed the electrical circuit between the cut pole of the RF blade and the tissue. Any volatile material entering the plasma slowly enough is vaporised and the perception is therefore of a tissue dissecting plasma.

The use of robotic equipment to assist surgery is increasing rapidly. Typically robot-assisted surgery involves the use of a robotic arm, which can be controlled directly or remotely by a surgeon to perform various movements or manipulations of a given surgical procedure. The robotic arm may have an end-effector at a distal end thereof. The end-effector may be or may carry a surgical instrument. Robotic-assisted surgical systems may be used in open and laparoscopic procedures.

It is known to use robot-assisted surgical systems in electrosurgical procedures. For example, the Da Vinci system manufactured by Intuitive Surgical allows for a generator to be integrated into a vision cart that is connectable to the patient cart that carries the robotic arms.

### SUMMARY OF THE INVENTION

At its most general, the present invention provides a robot-assisted surgical system in which apparatus for providing electrosurgical functionality is directly mountable on or integrated within a robotic arm. The apparatus may be a detachable module (referred to herein as a “capsule”), which may be movable between different robotic arms in the same environment. The apparatus may comprise a plurality of modules, each providing a different treatment modality. Depending on the procedure to be performed, a different module or combination of modules may be selected and mounted on one or more robotic arms.

The invention may provide a number of advantages. Firstly, by mounting the apparatus directly on the robotic arm, the means for generating energy for electrosurgery can be brought closer to the electrosurgical instrument. This facilitates the reduction or elimination of losses that can occur in conveying energy between a generator and the electrosurgical instrument. Secondly, providing the apparatus on the robotic arm avoids the need for a separate piece of operating suite furniture to house the electrosurgical generator. This may save space in the operating theatre. Thirdly, providing a modular set-up may enable each robotic arm in a multi-arm system to have the same functionality without the cost of independently configuring each arm for electrosurgery.

In a particularly advantageous arrangement, the invention may provide a detachable electrosurgical module for a robotic arm, where the electrosurgical module is powered by the robotic arm's internal power supply. For example, the robotic arm may have a DC supply available for control and movement of end effectors as well as for manipulation of the arm itself. The electrosurgical module may be configured to utilise the DC supply to generate other forms of energy, e.g. radiofrequency or microwave energy, to be supplied to an electrosurgical instrument held by the robotic arm. The arrangement may be advantageous because it obviates the requirement for a separate power supply to be provided on the robotic arm.

According to one aspect of the invention, there is provided an electrosurgical generator unit for a robot-assisted surgical system, the electrosurgical generator unit comprising: a housing configured to be detachably mountable on an articulated robotic arm of the robot-assisted surgical system; a signal generator contained within the housing, the signal generator being configured to generate an electrosurgical signal for use by the robot-assisted surgical system; and an energy delivery structure configured to couple the electrosurgical signal into the robot-assisted surgical system. This aspect of the invention provides a detachably and hence interchangeable unit that provides a localised source for an electrosurgical signal for use by a surgical instrument.

The electrosurgical generator unit may further comprise a controller contained within the housing and operatively connected to the signal generator. The controller may be configured to receive a control signal and to control the signal generator based on the received control signal. The control signal may preferably be transmitted through the robot-assisted surgical system. For example, the electrosurgical generator unit may further comprise an input portion that is communicably connectable to a control network of the robot-assisted surgical system, wherein the controller is configured to receive the control signal from the control network of the robot-assisted surgical system. This arrangement may avoid the need to provide a separate communication channel for the electrosurgical generator unit.

Alternatively or additionally, the controller may include a wireless communication module configured to receive the input control signal wirelessly.

The signal generator may be configured to provide an electrosurgical signal for use in one or more of a plurality of treatment modalities. For example, the electrosurgical signal may be a microwave signal or a radiofrequency (RF) signal having a power level suitable for causing tissue ablation at a distal end of the surgical instrument. In another example, the electrosurgical signal may be microwave energy or RF energy having a power selected to be suitable for measuring properties of tissue without causing any tissue damage. The signal generator may be capable of generating both an RF signal and a microwave signal, either separately or simultaneously. As explained in more detail below, the RF and/or microwave energy may be delivered in combination with a fluid (e.g. gas) to enable a plasma to be struck at the distal end of the surgical instrument.

Similarly, the RF and/or microwave energy may be delivered in combination with a cryofluid to enable cryoablation to be performed at the distal end of the surgical instrument.

In other examples, the signal generator may include a pulse generator configured to produce a waveform for the electrosurgical signal that makes it suitable for causing electroporation of tissue at the distal end of the surgical instrument.

The signal generator may be configured to produce other types of energy, e.g. ultrasound or the like.

The electrosurgical generator unit may itself have a modular configuration, wherein the housing may be configured to receive one or more detachable signal generator modules. In this arrangement, the electrosurgical generator unit may be adjustably configured to generate a electrosurgical signal for a desired purpose.

As mentioned above, the electrosurgical generator unit may further comprise a fluid supply and a fluid conduit configured to couple fluid from the fluid supply into the robot-assisted surgical system. In one example, the energy delivery structure for the electrosurgical signal and the fluid conduit may be contained in a common feed structure. The common feed structure may comprise a coaxial transmission line having an inner conductor separated from an outer conductor by a dielectric material, wherein the fluid conduit comprises a passageway formed within the inner conductor.

In a configuration where the electrosurgical generator unit is configured provide a measurement modality, it may further comprise a signal detector contained within the housing. The signal detector may be connected to the energy delivery structure and configured to sample a signal characteristic on the energy delivery structure. The signal characteristic may be an amplitude and/or phase of reflected power on the energy delivery structure, for example. The signal detector or the controller within the housing may be configured to generate a detection signal which is indicative of the signal characteristic. The detection signal may be output, e.g. returned through the control network of the robot-assisted surgical system, in a manner that is readable by a user, e.g. on a display on the control console.

The electrosurgical generator unit may include other types of measurement. For example, the electrosurgical generator unit may include an optical source and sensor unit for performing laser spectrometry. The electrosurgical generator unit may further include any one or more of a temperature sensing module and a radiometric tissue sensor.

In a particularly advantageous arrangement, the electrosurgical generator unit may be powered through an internal supply of the robot-assisted surgical system. The electrosurgical generator unit may thus comprise a power coupling unit configured to receive a power feed from the robot-assisted surgical system. The power feed may be a DC signal. The signal generator may be configured to generate the electrosurgical signal using the DC signal. The signal generator may be configured to adjust a voltage of the DC signal, e.g. using a linear or switched-mode regulator, to a level suitable for use.

Additionally or alternatively, the electrosurgical generator unit may have an independent power supply. For example, it may have a separate connection to a mains supply, or it may include a battery contained within the housing.

In one example, the signal generator may comprise a microwave source and an amplification unit coupled to the microwave source. In this example the electrosurgical signal may comprise a microwave signal. The amplification unit may include a power amplifier, and signal generator may be configured to extract from the DC signal both a drain voltage and a source voltage for the power amplifier.

In another aspect, the invention may provide an instrument holder for a robot-assisted surgical system. The instrument holder may comprise a body having: a proximal end that is mountable on and manipulable by an articulated arm of the robot-assisted surgical system; a distal portion configured to retain a surgical instrument; and an intermediate portion configured to receive an electrosurgical generator unit as set out above. For example, the intermediate portion may include a recess into which the electrosurgical generator unit can be plugged. The instrument holder and electrosurgical generator unit may have cooperating connectors to permit transfer of power and control signals therebetween. In particular, the instrument holder may be configured to couple an electrosurgical signal from the electrosurgical generator unit into the surgical instrument retained on the distal portion.

In a further aspect, the invention may provide a robot-assisted surgical system comprising: an articulated arm; an instrument holder mounted on a distal end of the articulated arm; an electrosurgical instrument mounted on the instrument holder; and an electrosurgical generator unit as set out above detachably mounted on the instrument holder, wherein the instrument holder is configured to couple an electrosurgical signal generated by the electrosurgical generator unit into the electrosurgical instrument.

The electrosurgical instrument may comprise an elongate probe having a proximal energy conveying structure and a distal tip. The instrument holder may be configured to couple the electrosurgical signal into the energy conveying structure for delivery to the distal tip.

The robot-assisted surgical system may further comprise a control console connected to the articulated arm via a control network, wherein the control console is configured to control the electrosurgical generator unit using a control signal transmitted via the control network.

Herein, in relation to a coaxial transmission line or other coaxial structure, the term “inner” means radially closer to the centre (e.g. axis) of the structure. The term “outer” means radially further from the centre (axis) of the structure.

The term “conductive” is used herein to mean electrically conductive, unless the context dictates otherwise.

Herein, the terms “proximal” and “distal” refer to position relative to the signal generator of the electrosurgical generator unit. In use, the proximal end is closer to the signal generator for providing the electrosurgical signal, whereas the distal end is further from the signal generator.

In this specification “microwave” may be used broadly to indicate a frequency range of 400 MHz to 100 GHz, but preferably the range 1 GHz to 60 GHz. Preferred spot frequencies for microwave EM energy include: 915 MHz, 2.45 GHz, 3.3 GHz, 5.8 GHz, 10 GHz, 14.5 GHz and 24 GHz. 5.8 GHz may be preferred. The device may deliver energy at more than one of these microwave frequencies.

The term “radiofrequency” or “RF” may be used to indicate a frequency between 300 kHz and 400 MHz.

### BRIEF DESCRIPTION OF THE DRAWINGS

Embodiments of the invention are described in detail below with reference to the accompanying drawings, in which:

FIG. 1 is an overall schematic system diagram of an robot-assisted electrosurgical system to which the present invention is applied;

FIG. 2 is a perspective view of an articulated robotic arm for electrosurgery that is an embodiment of the invention;

FIG. 3 is a schematic diagram of a instrument holder for an articulated robotic arm according to an embodiment of the invention;

FIG. 4 is a schematic diagram of a removable electrosurgery capsule for a robot arm according to an embodiment of the invention;

FIG. 5 is a schematic diagram of a microwave generation module suitable for use in the removable electrosurgery capsule of FIG. 4;

FIG. 6 is a schematic diagram of components for launching DC power and low power microwave energy into a common feed line for use in the removable electrosurgery capsule of FIG. 4;

FIG. 7 is a schematic circuit diagram showing a microwave generation module suitable for use in the removable electrosurgery capsule of FIG. 4;

FIG. 8 is a schematic diagram of another microwave generation module suitable for use in the removable electrosurgery capsule of FIG. 4; and

FIG. 9 is a schematic diagram of an electrosurgical instrument that can be handled by an articulated robotic arm in an embodiment of the invention.

### DETAILED DESCRIPTION; FURTHER OPTIONS AND PREFERENCES

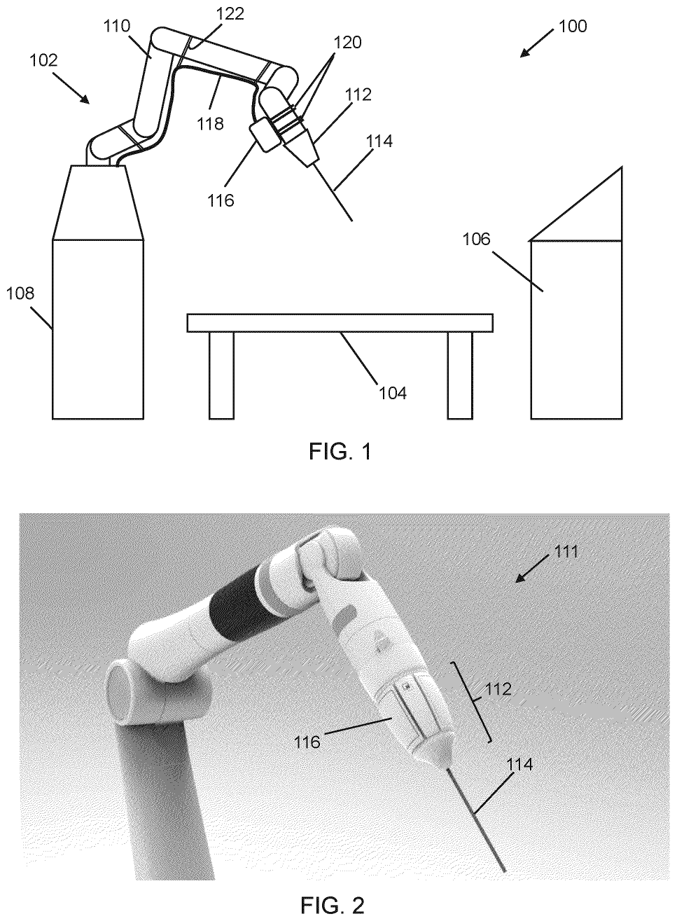

The present invention relates to the generation and use of electrosurgical instruments in the context of robot-assisted surgery. FIG. 1 is an overall schematic system diagram of an robot-assisted electrosurgical system 100 to which the present invention is applied. The system 100 comprises three main entities: a robotic surgical tool 102, an operating table 104, and a control console 106.

The operating table 104 provides a location for receiving a patient for a procedure with which the robotic surgical tool 102 can assist.

In this example, the robotic surgical tool 102 comprises a control column 108 having an articulated arm 110 extending therefrom. The control column 108 may support a plurality of articulated arms. A instrument holder 112 is mounted at a distal end of the articulated arm 110. The instrument holder 112 is configured to hold a surgical tool 114. In this example, the surgical tool 114 is depicted as a rigid elongate element, suitable for insertion into the patient's body e.g. using known laparoscopic techniques or the like. The articulated arm 110 allows the position and angle of the surgical tool 114 relative to the operating table 104 to be varied. The control column 108 may also be movable within the operating theatre environment.

The instrument holder 112 may comprise various ports that are connectable to the surgical instrument 114. For example, the instrument holder 112 may provide a link through which an end effector of the surgical instrument 114 can be controlled. The instrument holder 112 may also be used to deliver power or other substances (e.g. saline or the like) to the surgical instrument.

The control console 106, which is typically in the same room as the operating table 104 and robotic surgical tool 102, is normally separate from the robotic surgical tool 102 and is used to remotely control the articulated arm 110 and instrument holder 112. The articulated arm 110 may also be positioned manually.

In the invention, the robotic surgical tool 102 is provided with a detachable electrosurgery capsule 116 that is configured to generate and deliver, via the instrument holder 112, electrosurgical signals for use by the surgical instrument 114. In this example, the electrosurgery capsule 116 is secured to the articulated arm 110 by one or more suitable connections 120, e.g. straps or the like. However, in other examples discussed herein, the electrosurgery capsule 116 may be directly connectable to the instrument holder 112, e.g. as a plug-in module.

The electrosurgery capsule 116 may be a stand-alone unit for generation and delivery of signals suitable for use in electrosurgery.

As discussed in more detail below, the electrosurgery capsule 116 may be powered by an internal DC supply of the robotic surgical tool 102. That is, the robotic surgical tool 102 may be connected to a mains electricity supply in a standard manner (not shown). The control column 108 may include circuitry to transform the mains supply into a DC supply for use by the robot. The control column 108 may have a first DC supply for controlling movement of the articulated arm 110. Typically, the first DC supply may have a voltage of 24 V and permit current up to 2 A. The control column 108 may provide a second DC supply for use by or at the instrument holder 112. The second DC supply may have the same voltage (e.g. 24 V) or a lower voltage (e.g. 12 V) than the first DC supply, The second DC supply may have a more limited current supply (e.g. no more than 600 mA). The electrosurgery capsule 116 may utilise either the first or second DC supply. In the example shown in FIG. 1, the electrosurgery capsule 116 is connected to the control column 108 by a separate cable 118, which may be retained on the articulated arm 110 by one or more clips 122. The cable 118 may convey a DC signal from the first DC supply. Alternatively or additionally, the electrosurgery capsule 116 may be arranged to receive power via the same route as the instrument holder 112.

FIG. 2 is a perspective view of an articulated robotic arm 111 for electrosurgery that is another embodiment of the invention. Features in common with the system of FIG. 1 are given the same reference number. In this example, the articulated robotic arm 111 performs the same function as the articulated robotic arm 110 of FIG. 1. However, instead of having one or more electrosurgery capsules 116 attached to an outer surface thereof, the articulated robotic arm 111 has a instrument holder 112 provided with a recess configured to receive an electrosurgery capsule 116. The electrosurgery capsule 116 may be detachably mountable in the recess, e.g. to permit it to be readily exchanged for electrosurgery capsule that provides a different modality, or to permit the electrosurgery capsule 116 to be switched to another articulated robotic arm 111 on the same or a different control column.

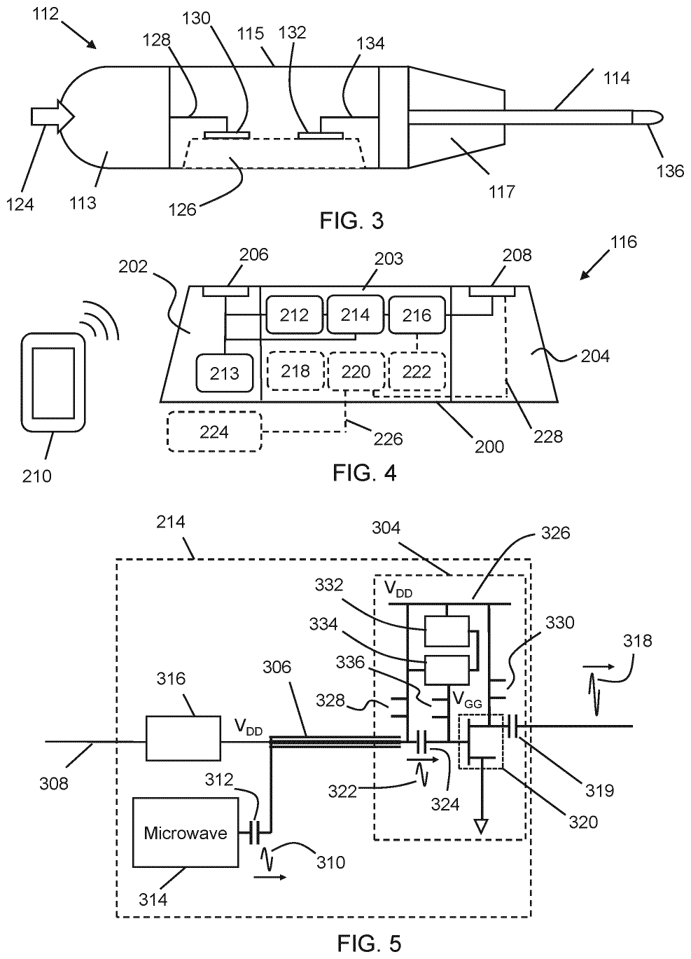

FIG. 3 is a schematic diagram of a instrument holder 112 for an articulated robotic arm 111 of the type shown in FIG. 2. The instrument holder 112 may have any suitable shape, although in the example it has a generally cylindrical form, extending along a longitudinal axis that is aligned with the surgical instrument 114 that extends from a distal portion 117 thereof.

The instrument holder 112 comprises a proximal portion 113 that is attached to (and may pivot on) a distal end of the articulated robotic arm. The proximal end 113 may be configured to receive a power input 124 that is conveyed through the articulated robot arm.

In this example, the instrument holder 112 comprises an intermediate portion 115 that has a recess 126 formed therein. There may be a plurality of recesses formed around a circumference of the intermediate portion. The instrument holder 112 may thus be configured to receive one or more electrosurgical capsules within the recesses 126. Where a plurality of electrosurgical capsules 116 are mounted, the instrument holder 112 may be configured to selectively connect any one or any combination of the electrosurgical capsules to the surgical instrument 114. The connection may operatively connect the electrosurgery capsule to a distal instrument tip 136, e.g. to permit an electromagnetic signal (e.g. comprising radiofrequency and/or microwave energy) to be conveyed to and delivered from the distal instrument tip 136. As discussed below, each electrosurgery capsule may be configured to generate an electromagnetic signal associated with a certain tissue treatment or measurement modality.

It is advantageous to generate the electromagnetic signal at the instrument holder 112 because it reduces the path length that the signal has to travel before reaching the distal instrument tip 136. This arrangement can therefore facilitate a reduction in power loss as the electromagnetic signal is conveyed. To achieve a given level of power at the distal instrument tip 136, the electrosurgical capsule may therefore be required to generate a lower power than a more distant generator. Or it may mean that higher powers may be achievable at the distal instrument tip 136 for a given power source.

Furthermore, having an electrosurgical generator on the instrument holder 112 obviates the requirement for a separate floor-based generator unit, which would otherwise occupy space within an operating theatre.

The intermediate portion 115 may further comprise means for interconnecting the electrosurgery capsule. For example, the recess 126 may have one or more input/output ports mounted on an internal surface thereof. In the example shown in FIG. 3, there is an input port 130 configured to deliver power (e.g. a DC signal) into the electrosurgery capsule. The input port 130 is connected to the proximal portion 113 by a suitable transmission line 128 that in turn is connected to the power input 124. Similarly, there is an output port 132 configured to deliver the electromagnetic signal (e.g. radiofrequency or microwave energy) from the electrosurgery capsule to the surgical instrument 114. The output port 132 may be connected to the distal portion 117 by a suitable transmission line 134 (e.g. a coaxial cable). The distal portion 117 may be configured with a suitable connector (e.g. QMA connector or the like) to connect the transmission line 134 to a energy conveying structure (e.g. another coaxial transmission line) within the surgical instrument 114 itself. An example of this is discussion below with reference to FIG. 9.

The surgical instrument 114 may be detachably mounted to the distal portion 117. The same instrument holder 112 may thus be used with a plurality of instruments. Moreover, in the invention the instrument holder 112 may be used with a plurality of different types of electrosurgery capsule. This enables a variety of combinations of instrument and energy modality to be used interchangeably at the same instrument holder.

FIG. 4 is a schematic diagram of a removable electrosurgery capsule 116 for a robot arm according to an embodiment of the invention. The electrosurgery capsule 116 may be receivable in a recess 126 of the type discussed above.

The electrosurgery capsule 116 comprises a rigid housing 200, which may be shaped to cooperate with a recess in the instrument holder of a robotic arm a manner that aligns the capsule appropriately. The electrosurgery capsule 116 includes an input portion 202 that is communicably connectable to a control network of the robot-assisted surgical system, e.g. via the instrument holder. The input portion 202 may also be configured to receive a power supply (e.g. an internal DC supply of the instrument holder), an operational portion 203 that houses various functional components or modules for generating and/or controlling an electromagnetic signal, and an output portion 204 for delivering the electromagnetic signal into the instrument holder, from where it is conveyed to an electrosurgical instrument held by the robotic arm.

In this example, the input portion 202 comprises an input connector 206 for receiving input control and power signals. The output portion 204 may comprise an output connector 208 for delivering a generated electromagnetic signal out of the electrosurgery capsule 116.

In the following description, the operational portion 203 of electrosurgery capsule 116 is presented as having a modular construction, in which various functional elements may be detachable or interchangeable depending on the desired output electromagnetic signal. Such a construction is advantageous in terms of flexibility of manufacture. However, it can be understood that the modular nature of the capsule component is not essential to the present invention. A capsule may be “hardwired” to provide a certain function, in which case the modules discussed below may be combined or include shared components.

In general, the electrosurgical capsule referred to herein is configured to produce electromagnetic (EM) radiation, such as radio frequency (RF) or microwave EM radiation, suitable for treating or measuring biological tissue.

In one example, control of the capsule (or of a plurality of capsules) is done using control signals delivered via a control network of the robot-assisted surgical system, e.g. using the control console, to a controller module 212 within the capsule. Control of the capsule may thus be centralised in a remote computing device 210, which may be control console of the robot-assisted system or a separate device. It may be preferable for control of the capsule to be achieved over a wired connection. However, in some examples, the capsule may be configured to communicate wirelessly. The remote computing device 210 may be a wireless computing device such as a laptop, a smartphone, a tablet computer, and the like. The remote computing device 210 is capable of wirelessly communicating with the electrosurgical capsule via a wireless communication channel so as to control the operation thereof.

In some examples, different optional modules may be combined together with core modules to provide a capsule with different electrosurgical capabilities.

Various electrosurgical modalities are presented below in the context of an robot-assisted surgical system for use in laparoscopic or endoscopic procedures involving the controlled delivery of EM energy, for example, RF and microwave energy. Such EM energy may be useful in the removal of polyps and malignant growths. However, it is to be understood that the aspects of the invention presented herein need not be limited to this particular application. Also, they may be equally applicable in embodiments where only RF energy is required, or where only RF energy and fluid delivery is required.

Returning to FIG. 4, the operational portion 203 of the electrosurgical capsule 116 in this example is configured as a modular system that includes a plurality of modules. The plurality of modules includes a controller module 212, a signal generator module 214, and a feed structure module 216. These may be the core modules of the operational portion 203. Additionally, the plurality of modules may include further optional modules: a signal detector module 218, a fluid feed module 220, and one or more additional signal generator modules 222. The optional nature of these modules is indicated in FIG. 4 by dashed lines.

The controller module 212 has a wireless communication interface operable to wirelessly communicate with the remote computing device 210, so as to receive instructions or data therefrom. The controller module 212 is operable to provide control commands based on the received data. For example, in one embodiment, the control commands may be all or part of the received data and, as such, the controller module 212 may forward the received data as the control commands. Also, the forwarding may involve removing part of the received data before forwarding. For instance, the received data may comprise a data packet which includes both the control commands and communication information, wherein the communication information is used to direct the data packet from its source (e.g. the remote computing device 210) to its destination (e.g. the controller module 212). The wireless communication channel 210 may be a direct channel between the remote computing device 210 and the controller module 212, but it may also be an indirect channel that includes, for example, one or more wired or wireless networks, such as, the Internet, a local area network, and/or a wide area network. In any case, the controller module 212 may remove or strip out this communication information (and, for example, any other information) such that only the control commands remain. Additionally or alternatively, however, the controller module 212 may include a processor (e.g. a microprocessor) which is coupled to the wireless communication interface so as to receive the received data. In use, the processor may generate the control commands based on the received data. That is, the received data may include none of the control commands, or only part of the control commands, such that the processor generates at least some of the control commands itself. It is to be understood the control commands are in a format that the module can understand and execute so as to perform one or more module functions.

The wireless communication interface of the controller module 212 and the remote computing device 210 enable the controller module 212 to wirelessly communicate with the remote computing device 210. Each wireless communication interface may be capable of communicating via one or more different protocols, such as, 3G, 4G, 5G, GSM, WiFi, Bluetooth® and/or CDMA. In any case, the controller module 212 and the remote computing device 210 may communicate with each other via the same protocol, such as, WiFi. Each wireless communication interface may include communication hardware for the transmission and reception of data signals, such as, a transmitter and a receiver (or a transceiver). Also, the communication hardware may include an antenna and an RF processor which provides an RF signal to the antenna for the transmission of data signals, and the receipt therefrom. Each wireless communication interface may also include a baseband processor, which provides data signals to and receives data signals from the processor. The precise construction of the wireless communication interface may vary between embodiments, as would be understood by the skilled person.

In an embodiment, the controller module 212 is operable to decrypt the data which is received at the wireless communication interface, for example, from the remote computing device 210. Also, the controller module 212 is operable to encrypt data which is transmitted by the wireless communication interface, for example, to the remote computing device 210. For instance, where the controller module 212 generates the control commands, the controller module 212 may transmit those generated control commands via the wireless communication interface to the remote computing device 210. Where the controller module 212 includes the processor, the encryption and decryption processes may be performed by the processor. Alternatively, the controller module 212 may include a separate encryption device for performing encryption and decryption. It is to be understood that any encryption protocol could be used, as would be known to the skilled person. However, given the electrosurgical nature of the invention, a medical encryption protocol may be preferable. An advantage of requiring that data be transmitted to/from the controller module 212 in encrypted form, is that a malicious party would find it more difficult or impossible to hack the electrosurgical system 200 so as to take control of the electrosurgical instrument 114. As such, system security and patient safety is improved.

In an embodiment, the controller module 212 includes a watchdog (or fault detection unit) for monitoring a range of potential error conditions which could result in the system 200 not performing to its intended specification. The watchdog is operable to generate an alarm signal when one of the potential error conditions occurs. For example, the watchdog may monitor a status of communication between the wireless communication module and the remote computing device 210, and a potential error condition may be a breakdown in communication between the controller module 212 and the remote computing device 210 for a duration above a preset threshold or time-period. For example, the watchdog may generate an alarm signal when the wireless communication module has been unable to communicate with the remote computing device 210 for more than ten seconds. It is to be understood that different time-periods could be used in different embodiments.

In an embodiment, the controller module 212 includes one or more sensors which monitor the operation of various parts of the system 200, and the watchdog may generate alarm signals when the outputs of these sensors moves outside of preset limits. For example, the controller module 212 may include one or more temperature sensors operable to generate temperature measurements based on a temperature of part of the controller module 212, such as, the processor or a memory of the controller module 212. The watchdog may then be operable to generate an alarm signal based on a comparison between the temperature measurements and one or more preset temperature limits, to indicate that the part is overheating. Additionally or alternatively, a different type of sensor (e.g. a voltage or current sensor) may be provided to monitor the operation of a fan which provides active cooling to the processor or memory, such that the watchdog generates an alarm signal if the sensor indicates that the fan has malfunctioned (e.g. it is using no voltage or current). Additionally or alternatively, a sensor may monitor a voltage level of a DC power supply of the controller module 212, and the watchdog may generate an alarm signal if the voltage level drifts out of a predetermined accepted range of operation. It is to be understood that the controller module 212 can contain different types of sensor which monitor the operation of different elements of the controller module, and the watchdog may monitor the outputs of these sensors and generate an alarm signal if any one of these outputs moves outside of preset limits. Additionally, the controller module 212 may contain sensors which monitor the operation of other modules, and the watchdog may monitor the outputs of these sensors and generate an alarm signal if any one of these outputs moves outside of preset limits.

The controller module 212 may handle an alarm signal in a number of different ways. For example, the controller module 212 may cause the watchdog to transmit the alarm signal via the wireless communication interface to the remote computing device 210. In this way, the remote computing device 210 can keep a record or log of when faults occur. Also, the watchdog may include in the alarm signal a reference to a type of fault to which the alarm signal relates such that the remote computing device 210 can include this information in the log. Also, the remote computing device 210 may externally control the response of the capsule based on the alarm signal. For example, the remote computing device 210 may send particular control commands to the controller module 212 based on the alarm signal, for example, so as to shut down the electrosurgical capsule 116 in a safe manner. In this way, the remoted computing device 210 may externally control the response of the electrosurgical capsule 116 based on the alarm signal. Additionally or alternatively, the controller module 212 may itself generate control commands based on the alarm signal. In this way, the controller module 212 may internally control the response of the electrosurgical capsule 116 based on the alarm signal. This internal control mechanism maybe particularly suitable for the loss of communication fault described earlier. On the other hand, the external control mechanism maybe particularly suitable for overheating faults described earlier. Therefore, a hybrid model may be adopted in which some faults are handled internally whereas some other faults are handled externally.

In an embodiment, where the controller module 212 includes the processor, the watchdog includes an independent processor (e.g. a microprocessor) so that the watchdog can confirm that the processor is functioning correctly, i.e. raise an alarm signal if the processor malfunctions (e.g. uses no voltage or current). Alternatively, the watchdog may be implemented in software which is executed by the processor of the controller module 212, i.e. no separate hardware processor may be included.

In summary, therefore, the controller module 212 receives data from the remote computing device 210 and, based on this received data, provides control commands to the signal generator module 214.

The capsule 116 may be powered by a feed from the robot-assisted surgical system, e.g. received via the instrument holder. For example, the input connector 206 may include a power coupling unit configured to receive a power feed. The power feed may be a DC supply from within the instrument holder, e.g. drawn from a DC supply used to manipulate the articulated arm.

The DC supply received at the input connector 206 may be used to power any one or more of the modules discussed herein. Alternatively or additionally, the capsule 115 may include a battery 213 contained within the housing 200. The battery 213 may provide an self-contained power supply, e.g. to supplement or provide a back-up to the power feed received through the input connector 206.

The signal generator module 214 is in communication with the controller module 212 so as to receive the control commands. For example, the signal generator module 214 may be coupled to the controller module 212 via a wired connection or cable. In use, the signal generator module 214 is operable to generate and control EM radiation based on the control commands to form an EM signal. The signal generator module may be any device capable of delivery EM energy for treatment of biological tissue. For example, the signal generator module 214 may be an RF signal generator module capable of generating and controlling RF EM radiation, for example, having a frequency of 100-500 KHz, or 300-400 MHz. Additionally, the RF signal generator module may include a bipolar or monopolar RF signal generator.

Alternatively or additionally, the signal generator module 214 may be a microwave signal generator module capable of generating and controlling microwave EM radiation, for example, having a frequency of 433 MHz, 915 MHz, 2.45 GHz, 5.8 GHz, 14.5 GHz, 24 GHz, or 30 to 31 GHz.

Alternatively, the signal generator module 214 may be an electroporation signal generator module capable of generating and controlling EM radiation having a low frequency, for example, 30 to 300 kHz.

The signal generator module 214 generates EM radiation based on the control commands. As such, example control commands may include an instruction for the signal generator module 214 to turn ON so as to generate EM radiation at its operational frequency, i.e. 433 MHz in the case of a 433 MHz microwave signal generator module. Also, the control commands may include an instruction for the signal generator module 214 to turn OFF so as to stop generating the EM radiation. Additionally or alternatively, the control commands could include other commands which specify other parameters of the EM radiation, for example, a duration that the signal generator module should generate EM energy, or a power (or amplitude) of the generated EM energy.

In an embodiment, the signal generator module 214 includes a pulse generator that is controllable by the controller module 212 based on the control commands to generate pulsed EM radiation from the EM radiation. Accordingly, the signal generator module 214 may be an electroporation signal generator module. For example, the EM radiation which is generated and controlled by the signal generator module 214 is operated on by the pulse generator so as to generate pulsed EM radiation which forms the EM signal that is received by the feed structure module 216. In this way, the signal generator module 214 is modified so as to provide a pulsed EM signal. The controller module 212 may control the pulse generator, via the control commands, to simply turn “ON” or “OFF” so that the signal generator module 214 generates an EM signal which is pulsed or continuous, respectively. Alternatively, the control commands may specify one or more pulse parameters, such as, a duty cycle, a pulse width (e.g. 0.5 ns to 300 ns), a rise time (e.g. Pico second or Nano second), or an amplitude (e.g. up to 10 kV). Additionally, the control commands may instruct the pulse generator to deliver a single pulse, a pulse train (e.g. number of pulses, or duration), or a burst of pulses (e.g. burst duration, number of pulses in burst, period between bursts).

In an embodiment, the signal generator module 214 includes one or more sensors which monitor the operation of different elements of the signal generator module 214 and send measurements to the controller module 212. As mentioned above, the controller module 212 (via the watchdog) can then compare these measurements to acceptable limits and generate an alarm signal if any one of these different elements develops a fault. For example, the signal generator module 214 may include a temperature sensor operable to generate temperature measurements based on a temperature of part of the signal generator module (e.g. an oscillator or an amplifier). The watchdog then generates the alarm signal based on a comparison between the temperature measurements and one or more preset temperature limits.

The signal generator module 214 may be powered by a signal received from the input connector 206 and/or by an internal battery 213. The signal may be a DC supply of the robotic arm on which the capsule is mounted. The DC signal may be used to power an amplification unit for increasing the power of a microwave signal in a manner discussed below with reference to FIGS. 5 to 8. It is desirable for the power supply of the electrosurgical capsule 116 either to be self-contained or to utilise power already available in the robotic arm. This arrangement obviates the need for a separate power feed.

In summary, the signal generator module 214 generates and controls EM radiation based on the control commands to form the EM signal. The frequency of the EM radiation is dependent on signal generator's type. The feed structure module 216 receives the EM signal from the signal generator module 214.

The feed structure module 216 is in communication with the signal generator module 214 so as to receive the EM signal. The feed structure module 216 provides an energy delivery structure configured to couple the EM signal into the surgical instrument via the robotic arm. The feed structure module 216 includes a signal channel which conveys the EM signal from the signal generator module 214 to an output port of the feed structure module 216. The output port is for outputting the EM signal to the output connector 208 and, as such, the output port may connect to a feed structure within the surgical instrument 114 via cooperating connectors. The feed structure module 216 may be coupled to the signal generator module 214 via a cable assembly which includes the signal channel. Also, the cable assembly may terminate in the output port. As such, in an embodiment, the feed structure module 216 may be a cable assembly connecting the signal generator module 214 to the output connector 208.

The optional modules 218, 220 and 222 will now be described in detail.

The signal detector module 218 is configured to sample a signal characteristic on the signal channel of the feed structure module 216, and to generate a detection signal which is indicative of the signal characteristic. For example, the signal generator module 214 may be an RF signal generator module, and the signal characteristic may be a voltage or a current present on the signal channel. Alternatively, the signal generator 214 may be a microwave signal generator module, and the signal characteristic may be a forward power or a reflected power present on the signal channel. In an embodiment, the signal generator module 214 may be configured to deliver a low power EM signal for the purposes of signal detection, and this low power signal may be referred to as a measurement signal, since it is generated for the purposes of measuring biological tissue at the distal end of the surgical instrument 114. Alternatively, an additional signal generator module 222, which will be described later, may be configured to provide the measurement signal. It is to be understood that the signal detector module 218 measures the signal channel and, as such, measures both signals emitted by the signal generator module 214, and signals which are reflected back to the feed structure module 216, for example, by biological tissue at a treatment site near to the distal end of the surgical instrument. Therefore, the measured signal characteristics are indicative of the biological tissue and, as such, the detection signal varies with tissue characteristics. In this way, the detection signal can be used to determine tissue characteristics (e.g. a tissue type).

In an embodiment, the controller module 212 is in communication with the signal detector module 218 so as to receive the detection signal. For example, the controller module 218 may be connected to the signal detection module 218 via a wired connection or cable. Also, the controller module 212 is operable to generate the control commands for the signal generator module 212 based on the detection signal. It is to be understood that the detection signal may be used (e.g. by the controller module 212 or remote computing device 210) to determine a characteristic of tissue at the treatment site, for example, it may indicate that the tissue is healthy or cancerous.

In use, the signal detector module 218 may provide a mechanism for the electrosurgical capsule 116 to dynamically respond to biological tissue being treated by the surgical instrument 114. For example, the signal generator module 214 may be a microwave signal generator module, and the measured signal characteristic may include forward and reflected power sampled on the microwave signal channel of the feed structure module 216. Based on the forward and reflected power, a return loss measured on the signal channel may be between −6 dB and −10 dB. This return loss may be indicative of a bleed. The controller module 212 (or the remote computing device 210), may determine this return loss, and further determine that it indicates a bleed, and then generate control commands for the microwave signal generator module to deliver a microwave EM signal with an appropriate (e.g. increased) power level and/or duty cycle until the bleed has been stemmed. Stemming of the bleed may be indicated by a change in the return loss measured from the reflected power. In an alternative embodiment, the signal generator module 214 may be an RF signal generator module, and the measured signal characteristic may include voltage (or current) sampled on the RF signal channel of the feed structure module 216. The indication of the onset of a bleed may also be provided by a change in measured voltage/current. As such, any cutting action of the RF signal generator module may be stopped so that the bleed can be addressed, for example, by a microwave signal generator module.

Additionally or alternatively, the controller module 212 is operable to transmit the detection signal from the wireless communication interface, for example, to the remote computing device 210. Accordingly, the remote computing device 210 is operable to generate control commands based on the detection signal, and then send those control commands to the controller module 212 for execution. It may be advantageous to have the remote computing device 210 generate the control commands because the remote computing device 210 may have more processing power than the controller module 212. Alternatively, it may be advantageous to have the controller module 212 generate the control commands because it may be significantly faster to transfer data from the controller module 212 directly to the signal generator module 214, rather than via the remote computing device 210. In an embodiment, both options may be available and the choice of whether to generate the control commands at the signal generator module 214 or at the controller module 212 is situation dependent. In any case, the detection signal may be used by the remote computing device 210 and/or controller module 212 to dynamically adjust system performance based on biological tissue being treated. These adjustments may improve treatment and patient safety.

In an embodiment, the feed structure module 216 further comprises a tuner connected to the signal channel for controlling the energy delivered by the EM signal. The tuner includes an adjustable impedance element that is controllable by the controller module 212 based on the detection signal. In an embodiment, the controller module 212 is connected to the feed structure module 216 (and tuner) via a wired connection or cable.

The tuner may function to promote efficient transfer of EM radiation into tissue. For example, information from the signal channel may be used to determine the adjustment of the adjustable impedance on the signal channel to provide dynamic power matching between the surgical instrument 114 and the tissue. This ensures efficient and controllable energy transfer between the electrosurgical capsule 116 and the biological tissue.

In an embodiment, the adjustable impedance element may be an adjustable reactance (e.g. capacitance or inductance). For example, the adjustable reactance may include a plurality of reactive elements, wherein each reactive element has a fixed reactance and is independently switchable into or out of connection with the signal channel according to a respective control command from the controller module 212. Alternatively, each reactive element may have a variable reactance that is independently controllable according to a respective control command from the controller module 212. Alternatively, the adjustable reactance may be provided by a variable capacitor and/or a variable inductor, and the controller module 212 includes a self-adjusting feedback loop arranged to generate a control command for setting the reactance of the variable capacitor or the variable inductor. Such embodiments may be particularly suitable where the signal generator module 214 is an RF signal generator module and the signal channel is an RF signal channel.

In another embodiment, the adjustable impedance element may be an impedance adjuster having an adjustable complex impedance that is controllable by the controller module 212. Such an embodiment may be particularly suitable where the signal generator module 214 is a microwave signal generator module and the signal channel is a microwave signal channel. In an embodiment, the signal detector module 218 or the feed structure module 216 includes one or more sensors which monitor the operation of different elements of the respective module and send measurements to the controller module 212. As mentioned above, the controller module 212 (via the watchdog) can then compare these measurements to acceptable preset limits and generate an alarm signal if any one of these different elements develops a fault.

The additional signal generator module 222 is analogous to the signal generator module 214 in the sense that the additional signal generator module 222 is operable to generate and control EM radiation based on control commands from the controller module 212 to form an EM signal. Further, in order to function with the additional signal generator module 222, the feed structure module 216 has one or more additional signal channels for coupling one or more additional signal generator modules 222 to the output port of the feed structure module 216. These additional signal channels may be included in the same physical structure (e.g. cable) as the previously described signal channel. In an embodiment, the feed structure 216 functions to combine together the EM signal from the signal generator module 214 with the EM signal from each additional signal generator module 222 such that they are all output from the output port to the distal end of the surgical instrument 114.

It is to be understood that the signal detector 218 may be configured to measure signal characteristics on each additional signal channel of the feed structure module 216, as described above. Also, the feed structure module 216 may include a tuner connected to each additional signal channel for controlling the energy delivered by the EM signal, as described above.

Any number of additional signal generator modules 222 may be provided. Further, each additional signal generator module 222 may generate EM radiation at a different frequency to the signal generator module 212 and to each other additional signal generator module 222.

In an embodiment, the signal channel for the signal generator module 214 and the signal channel for each additional signal generator module 222 may comprise physically separate signal pathways within the feed structure module 216. Also, the feed structure module 216 may include a signal combining circuit having one or more inputs, wherein each input is connected to a different one of the physically separate signal pathways. Also, the signal combining circuit has an output connected to a common signal pathway for conveying all the EM signals, separately or simultaneously, along a single channel to the output port. Stated differently, the signal combining circuit may provide a junction at which multiple EM signals arrive via separate signal paths from multiple different signal generator modules, and from which all the EM signals leave via the same signal path for delivery to the electrosurgical instrument 204.

In an embodiment, the signal combining circuit includes a switching device for selecting one or more of the EM signals to be connected to the common signal path. The switching device may be controllable based on control commands received from the controller module 212, for example, via a wired link between the controller module 212 and the feed structure module 216.

The provision of the additional signal generator modules 222a-n in combination with the aforementioned modifications to the feed structure module 216 mean that the electrosurgical capsule 116 can be adapted to provide different types of EM radiation to treat biological tissue. An advantage of this modular nature is that the functionality of the electrosurgical capsule 116 can increase so that the system can treat tissue in different ways so as to treat different conditions. Also, the functionality of the electrosurgical capsule 116 can be reduced so that the system is cheaper or smaller (e.g. more portable).

In an embodiment, each additional signal generator module 222 includes one or more sensors which monitor the operation of different elements of the additional signal generator module 222 and send measurements to the controller module 212. As mentioned above, the controller module 212 (via the watchdog) can then compare these measurements to acceptable preset limits and generate an alarm signal if any one of these different elements develops a fault.

The fluid feed module 220 includes a fluid feed structure 228 in fluid communication with a fluid port for outputting fluid to the surgical instrument 114. In this example, the fluid feed structure 228 delivers fluid to the output connector 208, where it may be conveyed to the surgical instrument via a suitable coupling within the instrument holder 112. The energy delivery structure and the fluid feed may be combined in a common feed structure. For example, the transmission line 134 within the instrument holder 112 may be configured as a combined fluid and energy feed to deliver both the fluid and the EM energy to the surgical instrument 114. The surgical instrument 114 may in turn comprise a fluid feed that transport fluid to the distal instrument tip 136.

A fluid supply 224 (e.g. a pressurised gas canister or the like) may be mounted on an external surface of the electrosurgery capsule 116. The fluid feed module 220 may connected to the fluid supply 224 by a feed conduit 226.

The fluid feed module 220 is controllable by the controller module 212 based on the control commands to supply and control a flow of fluid (e.g. gas or liquid) via the fluid feed structure 228 to the output connector 208. For example, the fluid feed module 220 may be connected to the controller module 212 by a wired connection or cable. The purpose of the fluid feed module 220 may be to provide fluid to the distal instrument tip 136. For example, the fluid may be a gas which is provided to the surgical instrument 114 for generating plasma for treatment of biological tissue. For example, non-thermal plasma may be used to sterilise tissue, for example, to kill bacteria present inside natural orifices or caused by foreign bodies introduced inside the body, i.e. metallic inserts. Also, thermal plasma may be used to cut tissue or perform surface coagulation, for example, for the treatment of ulcers on the surface of the tissue. The surgical instrument 114 may receive gas (from the fluid feed module 220) with either or both of RF energy or microwave energy (from the signal generator module 212 and one or more additional signal generator module 222) and use these components to emit either thermal plasma or non-thermal plasma. For example, for non-thermal plasma, the signal generator module 212 (acting as an RF signal generator module) may generate a high voltage state RF pulse (e.g. 400 V peak for 1 ms) to initiate the plasma using the gas, following which an additional signal generator module (acting as a microwave signal generator module) may generate a microwave pulse for a duration of 10 ms with a duty cycle of 10% and an amplitude of 30 W. On the other hand, for thermal plasma, the duty cycle may be increased to 60% and the amplitude to 60 W. In a general sense, when a flow of gas is present, the RF EM radiation is controllable to strike a conducting gas plasma and the microwave EM radiation is arranged to sustain the gas plasma. In an embodiment, the distal instrument tip 136 includes a bipolar probe which strikes the conducting gas between its two conductors. Being able to supply a combination of microwave and RF energy enables a high level of control over the thermal or non-thermal plasma produced at the distal instrument tip 136, as would be known to the skilled person, for example, in view of WO 2012/076844, which is incorporated herein by reference.

In an embodiment, the fluid feed module 220 may provide liquid (e.g. saline) to the distal instrument tip 136. In one embodiment, injection of fluid (saline or the like) is used to plump up the biological tissue at the treatment site. This may be particularly useful where the instrument is used to treat the wall of the bowel or the wall of the oesophagus or for protecting the portal vein or the pancreatic duct when a tumour or other abnormality located in close proximity, in order to protect these structures and create a cushion of fluid. Plumping up the tissue in this manner may help to reduce the risk of bowel perforation, damage to the wall of the oesophagus or leakage of from the pancreatic duct or damage to the portal vein, etc. This aspect may make it capable of treating other conditions where the abnormality (tumour, growth, lump, etc.) is close to a sensitive biological structure.

Also, the fluid feed module 220 may be configured to receive fluid from the surgical instrument 114. For example, fluid present at a treatment site at the distal instrument tip 136 may be sucked through the instrument fluid feed into the fluid feed module 220, for example, by a pump or other suction device in fluid communication with the fluid feed structure.

In an embodiment, the fluid feed module 220 includes a temperature control element controllable by the controller module 212 based on the control commands to vary a temperature of the fluid flow in the fluid feed structure. In this way, the fluid may be heated or cooled prior to being delivered to the distal instrument tip 136. The temperature control element may provide only heating or only cooling. The temperature control element may include a heater for heating the fluid. Also, the temperature control element may include a refrigerator for cooling the fluid.

In an embodiment, the signal generator module 212 (or an additional signal generator module 222) and the fluid feed module 220 may be used together to provide a cryoablation function. For example, the signal generator module 212 may be a microwave signal generator module, and the fluid feed module 220 may be configured to supply a tissue-freezing fluid to the surgical instrument 114. As such, the electrosurgery capsule 116 is capable of freezing biological tissue in a region around the distal instrument tip 136 and applying microwave energy to the frozen tissue. As water molecules in frozen tissue have reduced vibrational and rotational degrees of freedom compared to non-frozen tissue, less energy is lost to dielectric heating when microwave energy is transmitted through frozen tissue. Thus, by freezing the region around the distal end portion, microwave energy radiated from the distal end portion can be transmitted through the frozen region with low losses and into tissue surrounding the frozen region. This enables the size of the treatment area to be increased compared with conventional microwave ablation instrument (e.g. probes), without having to increase the amount of microwave energy delivered to the distal end portion. Once the tissue surrounding the frozen region has been ablated with microwave energy, the frozen region can be allowed to progressively thaw so that it will dissipate microwave energy and be ablated. The apparatus of the invention also enables various combinations of microwave energy and tissue freezing to be used to effectively ablate biological tissue.

The tissue-freezing fluid may be a cryogenic liquid or gas, and may be referred to herein as a cryogen. The term “cryogen” may refer to a substance which is used to produce temperatures below 0° C. Suitable cryogens include, but are not limited to liquid nitrogen, liquid carbon dioxide and liquid nitrous oxide. The fluid feed structure and instrument fluid feed structure may be provided with a thermal insulation layer made of a thermally insulating material and/or a vacuum jacket to prevent other parts of the apparatus from being cooled by the cryogen. This can also ensure that only tissue in the treatment zone is frozen, and that other parts of the patient which may be in close proximity to the cryogen conveying conduit are not affected by the cryogen.

In an embodiment, the fluid feed module 220 includes one or more sensors which monitor the operation of different elements of the fluid feed module 220 and send measurements to the controller module 212. As mentioned above, the controller module 212 (via the watchdog) can then compare these measurements to acceptable preset limits and generate an alarm signal if any one of these different elements develops a fault.

The structures for conveying fluid may be separate from the structures that are used to deliver electromagnetic signals. However, it may be desirable in some circumstances for these structures to be contained within the same physical structure, e.g. cable assembly. For example, it is advantageous to be able to use the same instrument to deliver fluid as delivers RF and/or microwave energy since deflation (e.g. due to fluid seepage or loss of insufflation air) may occur if a separate instrument is introduced into the region or during treatment. The ability to introduce fluid using the same treatment structure enables the level to be topped up as soon as deflation occurs. Moreover, the use of a single instrument to perform desiccation or dissection as well as to introduce fluid also reduces the time taken to perform the overall procedure, reduces the risk of causing harm to the patient and also reduces the risk of infection. More generally, injection of fluid may be used to flush the treatment region, e.g. to remove waste products or removed tissue to provide better visibility when treating. This may be particularly useful in endoscopic procedures. In an embodiment, the feed structures of the invention include those disclosed in WO 2012/095653, which is incorporated herein by reference.

The electrosurgery capsule 116 shown in FIG. 4 illustrates one specific embodiment a modular arrangement. However, it may be understood that the functionality of the electrosurgery capsule 116 can be changed by adding or removing certain optional modules to the core modules. As mentioned above, the core modules are the controller module 212, the signal generator module 214, and the feed structure module 214. These core modules provide mechanisms for controllably generating an EM signal for treating biological tissue, and for delivering that EM signal to an electrosurgical instrument. The EM signal may be any type of electromagnetic signal, such as, RF or microwave. Furthermore, this core functionality can be supplemented in different ways to provide additional functionality. For instance, a signal detector module 218 may be provided to monitor a state of the tissue to determine a tissue characteristic or so that treatment (e.g. the EM signal) can be adapted to the tissue. The signal detector module 218 may use the signal generator module 212 to provide a measurement signal (e.g. a low power microwave signal); however, a separate additional signal generator module 222 may be used to generate the measurement signal. Additionally or alternatively, one or more additional signal generators 222 maybe provided such that the capsule can deliver EM signals having multiple different frequencies. In one example, both RF and microwave EM signals may be provided by the capsule. In another example, multiple different frequencies of microwave EM signal may be provided. Furthermore the feed structure module 216 can be configured to deliver one or more of the multiple different EM signals separately or simultaneously to the surgical instrument 114. Finally, a fluid feed module 220 may be provided to deliver/receive fluid to/from the treatment site. For example, gas may be provided in combination with RF or microwave energy in order to generate plasma. Alternatively, a tissue freezing fluid may be delivered with EM energy in order to perform cryoablation. Further, liquid may be extracted (e.g. by suction or pumping) from the treatment site.

The electrosurgery capsule 116 of FIG. 4 includes a remote computing device 210 which communicates wirelessly with the controller module 212. The controller module 212 communicates with each other module of the electrosurgery capsule 116, and can control each other module via control commands. For example, the controller module 212 may issue a control command to the signal generator module 214 to generate an EM signal. The controller module 212 may issue a control command to the feed structure module 216 to tune the signal channel by varying its adjustable impedance element. In any case, as described above, the controller module 212 may generate the control commands itself but it may also simply forward control commands which it receives from the remote computing device 210. Therefore, in an embodiment, control of the system 200 is centralised in the remote computing device 210, and the controller module 212 may only forward control commands to the modules and may not generate or process data received from the remote computing device 210. However, in another embodiment, the controller module 212 may perform at least some of the control of the capsule 116 and, as such, control of the capsule 116 may be shared between the remote computing device 210 and the controller module 212. It is to be understood that in this hybrid arrangement, control of the capsule 116 may still be centralised in the remote computing device 210, and the controller module 212 may supplement this control only in certain circumstances, for example, when communication between the remote computing device 210 and the controller module 212 breaks down. Alternatively, control of the capsule 116 may be centralised in the controller module 212, and the remote computing device 210 may supplement this control only in certain circumstances, for example, where user input is required. In summary, therefore, overall control of the capsule 116 may be controlled by either or both of the remote computing device 210 and the controller module 212.

As mentioned above, the electrosurgery capsule 116 may be powered entirely by a local battery or a DC supply from the robot arm on which it is mounted. In other words, the electrosurgery capsule need not require a dedicated connection to an external main supply. This may be desirable, as it obviates the need to consider means for isolating the mains supply from the surgical instrument and, ultimately, the patient. FIGS. 5 to 8 illustrate circuits in which a DC signal can be utilised in an amplification unit for a microwave signal generation module that is suitable for use in an electrosurgery capsule 116 as discussed herein.

FIG. 5 is a schematic diagram of a microwave generation module that may be used as a signal generation module 214 of the type shown in the arrangement depicted in FIG. 4.

The signal generation module 214 receives as an input DC power on DC supply line 308. The DC power is received in a signal conditioning unit 316, which functions to launch a DC signal into a common transmission line structure 306. The DC signal having a voltage V_{DD} of 24 V, for example.

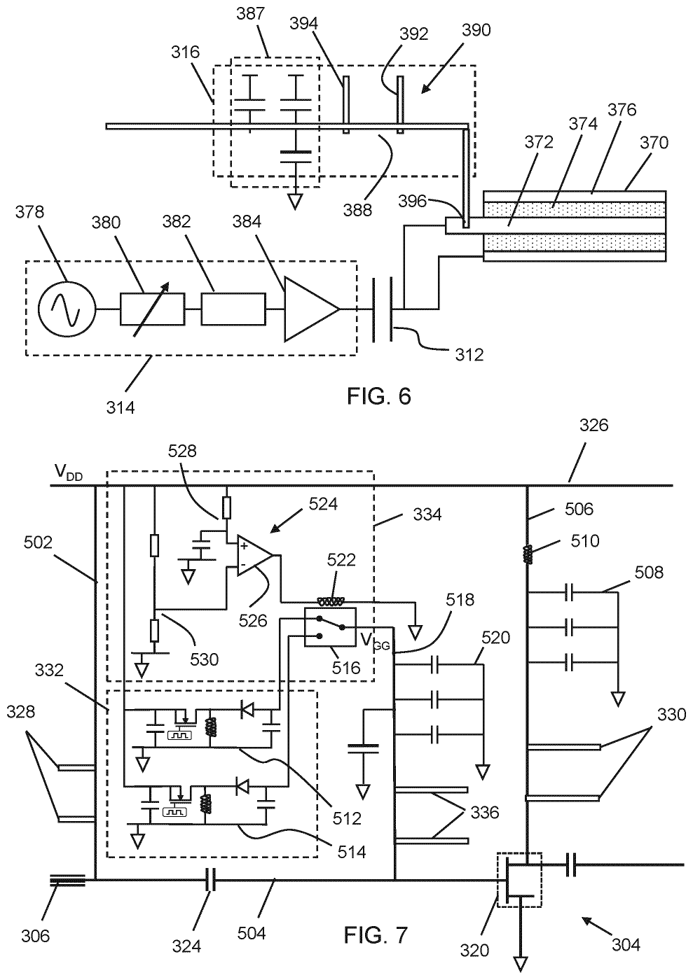

The signal generation module 214 further comprises a microwave source 314 that is configured to launch a microwave signal 310 into the common transmission line structure 306, which in this example is a coaxial transmission line. The microwave signal generator 314 is described below with reference to FIG. 6. The microwave signal 310 from the microwave signal generator 314 is coupled to the coaxial transmission line via a capacitor 312, which acts as a DC isolation barrier to prevent the DC signal from leaking into the microwave signal generator 314.

Advantageously, the DC signal is launched on an inner conductor of the coaxial transmission line that carries the microwave signal 310. However, in other examples an independent elongate conductor (e.g. wire) for conveying the DC signal may be provided.

The transmission line structure 306 conveys the DC signal and microwave signal 310 to an amplification unit 304, which functions to amplify the microwave signal 310 to a power level suitable for treatment. The amplified microwave signal 318 is output by the amplification unit 304, whereupon it is coupled via capacitor 319 to the feed structure module 216, from which it is delivered to a surgical instrument 114. The capacitor 319 operates as a DC barrier between the feed structure module 216 and the amplification unit 304 to prevent the DC signal from reaching the instrument.

The amplification unit 304 includes a power amplifier 320, e.g. a power MOSFET or the like. The power amplifier 320 receives as an input the microwave signal 322 output from the coaxial transmission line. The input to the power amplifier 320 is protected from the DC signal by a capacitor 324.

The amplification unit 304 is arranged to separate the DC power from the microwave signal, and apply it across the power amplifier 320. The amplification unit 304 may include a voltage rail 326 to which the DC signal (V_{DD}) is applied. The microwave signal 322 may be blocked from the voltage rail 326 by filtering arrangement 328, which may comprises a pair of quarter wave stubs as discussed in more detail below. Similarly a filtering arrangement 330 may also be disposed on the connection between the voltage rail 326 and power amplifier 320 to prevent microwave energy from leaking out on the voltage rail 326 from the power amplifier 320.

The amplification unit 304 further comprises a gate voltage extraction module 332 that operates to derive from the DC signal a bias voltage V_{GG} to be applied to the gate of the power amplifier 320. The gate voltage extraction module 332 may include a DC-DC converter, which down-converts the DC signal voltage to a suitable level for the power amplifier 320.

The distal amplification portion 304 may further comprise a gate control module 334 for controlling application of the gate voltage to the power amplifier 320. As discussed in more detail below, the gate control module 334 may operate to switch between two bias voltage states, which correspond respectively to an ON (conducting) and OFF (non-conducting) condition for the power amplifier 320. The gate control module 334 may operate to introduce a time delay between application of the DC signal across the power amplifier 320 (i.e. as its drain voltage) and application of a bias voltage to turn on the power amplifier 320 in order to ensure a smooth initialisation of the amplification process.

A filtering arrangement 336 may be disposed on the connection between the gate control module 334 and the gate of the power amplifier 320 to prevent microwave energy from leaking into the gate control module 334 from the power amplifier 320.

Detailed structures for the gate voltage extraction module 332 and gate control module 334 are discussed below with reference to FIG. 7.

In use, the microwave generation module thus performs the amplification of a low power microwave signal to a power level suitable for treatment. The amplified power level may be one or more orders of magnitude higher than the power level output from the microwave source 314, e.g. 10 W or more.

FIG. 6 is a schematic diagram showing further details of the signal conditioning unit 316 and the microwave source 314, which are configured to launch a microwave signal and a DC signal into a proximal end of a coaxial transmission line 370. Features in common with FIG. 5 are given the same reference number and are not described again. The coaxial transmission line 370 comprises an inner conductor 372 separated from an outer conductor 376 by a dielectric material 374. The coaxial transmission line 370 may be a Sucoform cable manufactured by Huber+Suhner, for example.

FIG. 6 shows components for the microwave signal generator 314. In this example, microwave signal generator 314 has a microwave frequency source 378 followed by a variable attenuator 380, which may be controlled by a controller module 212 of the electrosurgery capsule. The output of the variable attenuator 380 is input to a signal modulator 382, which may also be controlled by the controller module 212, e.g. to apply a pulsed waveform to the microwave signal. The output from the signal modulator 382 is input to a drive amplifier 384 to generate the microwave signal at the desired power level for input to the amplification unit 304. The microwave signal is coupled to the coaxial transmission line 370 via a capacitor 312.

The signal conditioning unit 316 for the DC signal comprises a section of microstrip transmission line 388 on which a low pass filter 390 is provided to prevent back transmission of the microwave signal into input connector from which the DC signal is received. The low pass filter 390 comprises a pair of quarter wave stubs 392, 394 on the microstrip transmission line 388. A first stub 392 is located at a half wavelength

$$( i . e . \frac{n λ}{2} )$$

distance from a connection point 396 to the inner conductor 372 of the coaxial transmission line 370, where λ is the wavelength of the microwave signal on the microwave transmission line 388, and n is a whole number equal to 1 or more. This ensure that the base of the first quarter wave

$$( i . e . \frac{( 2 n - 1 ) λ}{4} )$$

stub 392 is at a short circuit condition, so that the other end of the quarter wave stub 392 is in an open circuit condition. A second quarter wave stub 394 is spaced from the first stub by a half wavelength

$$( i . e . \frac{n λ}{2} )$$

distance. The signal conditioning unit 316 may further comprise a set of capacitors 387 connected in shunt to the transmission line that conveys the DC signal in order to remove any other unwanted AC element on the DC signal path.

FIG. 7 is a schematic circuit diagram showing an amplification unit 304 for an embodiment of the invention. Features in common with the previous drawings are given the same reference number and are not described again.

In this example, a distal end of the transmission line structure 306 provides an input to the amplification unit 304. The transmission line structure 306 may include the coaxial transmission line 370 discussed above, which conveys both the microwave signal and the DC signal. The amplification unit 304 splits the microwave signal from the DC signal using filters. The DC signal passes to the DC rail 326 via a first connection line 502, which has a low pass filter comprising a pair of quarter wave stubs 328 arranged to prevent passage of the microwave signal.

The pair of stubs 328 may be fabricated on a microstrip transmission line. A first stub is located at a half wavelength

$$( i . e . \frac{n λ}{2} )$$

distance from a connection point to the inner conductor of the coaxial transmission line, where λ is the wavelength of the microwave signal on the microwave transmission line, and n is a whole number equal to 1 or more. This ensure that the base of the first quarter

$$( i . e . \frac{( 2 n - 1 ) λ}{4} )$$

stub is at a short circuit condition, so that the other end of the quarter wave stub is in an open circuit condition. A second quarter wave stub is spaced from the first stub by a half wavelength

$$( i . e . \frac{n λ}{2} )$$

distance.

Meanwhile the microwave signal passes to a power amplifier 320 along connection line 504, where it becomes an input signal to be amplified. The connection line 504 may be a microstrip transmission line or the like. The connection line 504 includes a capacitor 324 through which the microwave signal is coupled but which blocks the DC signal. The capacitor 324 therefore isolates the power amplifier 320 from any DC component conveyed from the coaxial transmission line 370.

A connection line 506 connects the voltage rail 326 to the power amplifier 320 to apply a voltage of the DC signal across the power amplifier 320 (i.e. as a drain supply). To prevent microwave energy from leaking out of the power amplifier 320 on the connection line 506, a pair of quarter wave stubs 330 are arranged as a low pass filter. The pair of stubs 330 may be arranged in a similar manner to the stubs 328, albeit with respect to a connection point between the connection line 506 and the power amplifier 320.

The connection line 506 further comprises a set of capacitors 508 connected in shunt to the connection line that conveys the DC signal in order to remove any other unwanted AC element on the DC signal path.

The connection line 506 further comprises an inductor 510 connected in series between the power amplifier 320 and voltage rail 326. The inductance further inhibits leakage of AC signals onto the voltage rail 326.

Each of the connection lines discussed above may be implemented as a suitable transmission line for conveying DC or microwave signals as appropriate. Microstrip lines, e.g. on a flexible substrate that can be wrapped into a compact configuration are a suitable example.

In this embodiment, the amplification unit 304 is configured to extract a bias voltage V_{GG} for the power amplifier from the voltage rail 326. The voltage rail 326 may be at a relatively high voltage, e.g. 24 V or similar, whereas the bias voltage for the power amplifier 320 may need to be an order of magnitude lower. To obtain the bias voltage, the distal microwave amplification module 304 includes a gate voltage extraction module 332. The gate voltage extraction module 332 functions as a DC-DC converter, and in this embodiment it is implemented as a pair of parallel buck converters 512, 514, each of which is configured to output a different voltage, so that the bias voltage can be switched between two different states.

Each buck converter 512, 514 is connected to the voltage rail 326 to provide an input voltage. The values of the capacitance and inductance within each buck converter 512, 514 are selected to transform the input voltage to a desired output voltage. The output voltages may be selected based on the operational characteristic of the power amplifier. In this example, the buck converters 512, 514 are configured to generate a negative output voltage by using a diode to control an appropriate current flow direction in each converter. This means the output voltages (bias voltages) can be set close to the point in its characteristic where the power amplifier enters a conducting state.

For example, a first buck converter 512 may be configured to output a bias voltage that lies in a non-conducting part of the power amplifier characteristic, e.g. −6 V. A second buck converter 514 may be configured to output a bias voltage that lies in a conducting part of the power amplifier characteristic, preferably just beyond a transition to the conducting state, e.g. −2 V.

The outputs from the pair of buck converters 512, 514 are connected to respective input poles of a switch 516 that forms part of a gate control module 334. An output of the switch 516 is connected to a connection line 518 which in turn is connected to connection line 504 to provide the bias voltage from the gate voltage extraction module 332 to a gate of the power amplifier 320.

To prevent microwave energy from leaking out of the power amplifier 320 on the connection line 518, a pair of quarter wave stubs 336 are arranged as a low pass filter. The pair of stubs 336 may be arranged in a similar manner to the stubs 328, albeit with respect to a connection point between the connection line 518 and the connection line 504.

The connection line 518 further comprises a set of capacitors 520 connected in shunt to the connection line 518 that conveys the bias voltage in order to remove any other unwanted AC element on the bias voltage signal path.

The gate control module 334 operates to apply a required bias voltage to the gate of the power amplifier 320. The gate control module 334 thus effectively operates to selectively activate the power amplifier 320. In this example, the gate control module 334 functions to control the switch 516 that selects the buck converter 512, 514 to provide the bias voltage to the power amplifier 320. The switch 516 may be controlled by an inductor 522 that is energised upon application of the DC signal to the voltage rail 326. The switch 516 may thus adopt a default (e.g. OFF) configuration when the inductor 522 is not energised. In this configuration, the switch 516 connects the buck converter with the non-conducting voltage level (e.g. −6 V) to the power amplifier. When the inductor 522 is energised, the switch adopts an activated (e.g. ON) configuration, in which the buck converter with the conducting voltage level (e.g. −2 V) is connected to the power amplifier.

In this embodiment, the gate control module 334 includes a ‘soft-start’ circuit 524 for the power amplifier 320, which acts to delay the change of state of the switch by smoothly increasing the voltage applied to the inductor 522. An advantage of this arrangement is that it enables the drain voltage across the power amplifier 320 to reach a steady state before a bias voltage to activate the power amplifier is applied. The ‘soft-start’ circuit 524 is implemented using a comparator 526 which generates an output to the inductor 522 based on a difference between a varying first input from an RC circuit 528 and a fixed input from a voltage divider circuit 530.

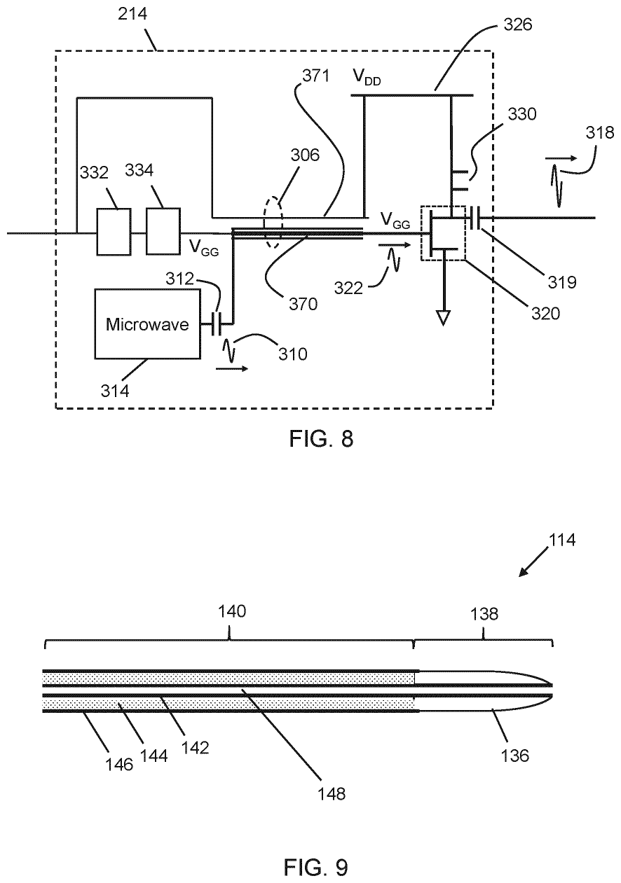

FIG. 8 is a schematic diagram showing another example of a signal generator module 214 that is configured as a microwave amplification apparatus. Features in common with FIG. 5 are given the same reference number and are not described again.

The signal generator module 214 in FIG. 8 differs from that in FIG. 5 in that the gate voltage is generated at the proximal end and transferred as a secondary DC signal through the transmission line 306.

The received DC signal (e.g. having voltage V_{DD}) in this arrangement may be conveyed to the amplification unit 304 by a dedicated transmission line 371. In the amplification unit 304, a distal end of the transmission line 371 is coupled to the drain of the power amplifier 320 through a low pass filter 330 that may be of the type described above. The dedicated transmission line 371 may be connected directly to the drain via the low pass filter, or may be connected via voltage rail 326 as shown in FIG. 8.

In this arrangement, the means for generating the bias voltage for the power amplifier may be located at a proximal end of the transmission line 306. For example, a gate voltage extraction module 332 may be configured for operation in the same way as described above, and a gate control module 334 may be provided for controlling the bias voltage that is supplied to the transmission line 306.

In this example, the bias voltage is conveyed to the distal portion along an inner conductor of a coaxial transmission line 370 in the cable assembly 306. The coaxial transmission line 370 is also used to convey the microwave signal 310 from the microwave signal generator 314.

In some examples, the dedicated line 371 for the DC signal may be an additional conductive layer formed around an outer conductor of the coaxial transmission line 370 and separated therefrom by an insulating layer, e.g. effectively to form a signal triaxial cable. In this example it may be desirable to include a low filter in the amplification unit 304 at the point where the DC signal is separated from the coaxial transmission line 370 to avoid the microwave signal from leaking on to the voltage rail 326.

FIG. 9 is a schematic cross-sectional view through an electrosurgical instrument 114 that can be handled by an articulated robotic arm in an embodiment of the invention. The electrosurgical instrument 114 may be connectable to the electrosurgery capsule 116 via the articulated robotic arm in a manner discussed above. The electrosurgical instrument 114 may be arranged or configured to deliver EM radiation from a distal instrument tip (or distal assembly) 136 in order to treat biological tissue located at a treatment site at or near to the distal assembly. The electrosurgical instrument 114 may be any device which in use is arranged to use EM energy (e.g. RF energy, microwave energy) for the treatment of biological tissue. The electrosurgical instrument 114 may use the EM energy for any or all of resection, coagulation and ablation. For example, the instrument 114 may be a resection device, a pair of microwave forceps, or a snare that radiates microwave energy and/or couples RF energy, and an argon beam coagulator.

The electrosurgical instrument 114 includes an instrument feed structure 140 for conveying EM radiation (e.g. an EM signal) to a distal end 138. In this example, the feed structure 140 is a coaxial transmission line formed from an inner conductor 142 that is separated from an outer conductor 146. The inner conductor 142 is hollow to define a passageway 148 for delivery of fluid.

1. A robot-assisted surgical system (100) comprising:
an electrosurgical generator unit;
an electrosurgical instrument; and
a robotic surgical tool comprising;
an articulated arm;
an instrument holder comprising a body having:
a proximal portion that is mountable on and manipulable by the articulated arm, the proximal portion being configured to receive a power input that is conveyed through the articulated arm;
a distal portion configured to retain the electrosurgical instrument; and
an intermediate portion configured to receive the electrosurgical generator unit,
wherein the electrosurgical generator unit is detachably mountable on the instrument holder and comprises:
a housing;
an input connector having a power coupling unit configured to receive a power feed via the instrument holder;
a signal generator contained within the housing, the signal generator being configured to generate an electrosurgical signal; and
an energy delivery structure configured to couple the electrosurgical signal into an output port of the intermediate portion, from where it is deliverable to the electrosurgical instrument via a transmission line disposed within the instrument holder.

2. The robot-assisted surgical system of claim 1, wherein the intermediate portion comprises a recess, and wherein the housing is detachably mountable in the recess.

3. The robot-assisted surgical system of claim 1, wherein the intermediate portion comprises a plurality of recesses, each recess being configured to receive a respective electrosurgical generator unit, and wherein the intermediate portion is configured to couple one or more electrosurgical signals from the electrosurgical generator units into the electrosurgical instrument.

4. The robot-assisted surgical system of claim 1, wherein the electrosurgical generator unit further comprises a controller contained within the housing and operatively connected to the signal generator, wherein the controller is configured to receive a control signal and to control the signal generator based on the received control signal.

5. The robot-assisted surgical system of claim 4, wherein the electrosurgical generator unit further comprises an input portion that is communicably connectable to a control network of the robot-assisted surgical system, wherein the controller is configured to receive the control signal from the control network of the robot-assisted surgical system.

6. The robot-assisted surgical system of claim 4, wherein the controller includes a wireless communication module configured to receive the input control signal wirelessly.

7. The robot-assisted surgical system of claim 1, wherein the electrosurgical generator unit further comprises a fluid supply and a fluid conduit (226) configured to couple fluid from the fluid supply into the robot-assisted surgical system.

8. The robot-assisted surgical system of claim 7, wherein the energy delivery structure and the fluid conduit are contained in a common feed structure, wherein the common feed structure comprises a coaxial transmission line having an inner conductor separated from an outer conductor by a dielectric material, wherein the fluid conduit comprises a passageway formed within the inner conductor.

9. The robot-assisted surgical system of claim 1, wherein the electrosurgical generator unit further comprises a signal detector contained within the housing and connected to the energy delivery structure, wherein the signal detector is configured to sample a signal characteristic on the energy delivery structure, and to generate a detection signal which is indicative of the signal characteristic.

10. The robot-assisted surgical system of claim 1, wherein the power feed is a DC signal, and wherein the signal generator is configured to generate the electrosurgical signal using the DC signal.

11. The robot-assisted surgical system of claim 1, wherein the electrosurgical generator unit further comprises a battery contained within the housing, wherein the battery is configured as an internal power supply for the electrosurgical generator unit.

12. The robot-assisted surgical system of claim 1, wherein the signal generator comprises a microwave source and an amplification unit coupled to the microwave source, and wherein the electrosurgical signal comprises a microwave signal.

13. The robot-assisted surgical system of claim 1, wherein the signal generator comprises a radiofrequency (RF) signal generator, and wherein the electrosurgical signal comprises an RF signal.

14. The robot-assisted surgical system of claim 1, wherein the electrosurgical instrument comprises an elongate probe having a proximal energy conveying structure and a distal tip, wherein the instrument holder is configured to couple the electrosurgical signal into the proximal energy conveying structure for delivery to the distal tip.

15. The robot-assisted surgical system of claim 14, further comprising a control console connected to the articulated arm via a control network, wherein the control console is configured to control the electrosurgical generator unit using a control signal transmitted via the control network.
